(19) United States
(12) Plant Patent Application Publication
(10) Pub. No.: US 2015/0067939 P1
(43) Pub. Date: Mar. 5, 2015

(54) Salvia greggii plant named 'Ruby Slippers'

(50) ;

(71) Applicant: Nicholas Benoit Shipley, Tucson, AZ (US)

(72) Inventor: Nicholas Benoit Shipley, Tucson, AZ (US)

(21) Appl. No.: 13/987,784

(22) Filed: Sep. 3, 2013

(51) Int. Cl. A01H 5/00

(52) U.S. Cl. PLT/475

(57) ABSTRACT

A new and distinct Salvia greggii plant named Ruby Slippers is characterized by vivid red flowers, compact form, long bloom period and high tolerance to desert heat.

### LATIN NAME

Salvia greggii

### VARIETAL DENOMINATION

Ruby Slippers

### BACKGROUND OF THE INVENTION

Salvia greggii, a member of the plant family Lamiaceae, is native to central and southwest Texas and south through the highlands of the Chihuahuan Desert in Mexico. Salvia greggii, which is also known as Autumn Sage, produces shrubby growth, ultimately reaching 1-3 ft tall×2-4 feet wide. Plants flower almost year around in mild climates, producing ¼-1 inch long tubular flowers which attract hummingbirds. Flower color varies from the typical reds found in the wild to white, pink, yellow and shades of purple in cultivated varieties.

Autumn sage has been cultivated in Texas as early as 1885, becoming popular in the southwestern United States as a landscape shrub about 85 years later and subsequently as a landscape shrub and as an annual flowering bedding plant worldwide. Many cultivars and hybrids have been produced, many including parentage from the closely related Salvia microphylla, which has a much larger adaptational range as well as more variability as a species.

The present invention relates to a new and distinct cultivar of Salvia greggii named ‘Ruby Slippers’. The cultivar originated as a seedling selection from offspring of the unpatented cultivar ‘Lipstick’ planted in 2008. The resulting plant has a different colored flower from its parent, as well as improved heat tolerance and a more compact growth form and is the object of this application.

### SUMMARY OF THE INVENTION

Among the features that distinguish the new Salvia greggii cultivar from all other available and commercial varieties of Salvia greggii known to the inventor is the following combination of characteristics: compact form, long bloom season, heat tolerance exceeding that of the unpatented cultivar ‘Lipstick’ and vivid red (overall) flowers (Royal Horticultural Society 50A-B). Mature plants reach about 2.5 feet high by 3.5 feet wide. Flowers are produced in pairs on terminal spikate racemes.

The propagation procedure is as follows: Softwood cuttings containing one node with leaves and about 3″ long are prepared and wetted with Dip & Grow™ and planted in potting medium in a misting greenhouse with bottom heat in the cool season. During the warm season bottom heat is not needed. Rooting is normally completed in 3-4 weeks.

The foregoing characteristics and distinctions come true to form and are established and transmitted through succeeding propagations. The present invention has not been evaluated under all possible environmental conditions, such that the phenotype may vary with variations in environment without a change in the genotype of the plant.

### BRIEF DESCRIPTION OF THE DRAWINGS

The accompanying photographs illustrate Salvia greggii ‘Ruby Slippers’ growing near Tucson, Ariz., depicted in color as nearly correct as it is possible to make in a color illustration of the character.

FIG. 1 illustrates a typical Salvia greggii ‘Ruby Slippers’ at about age 2 years.

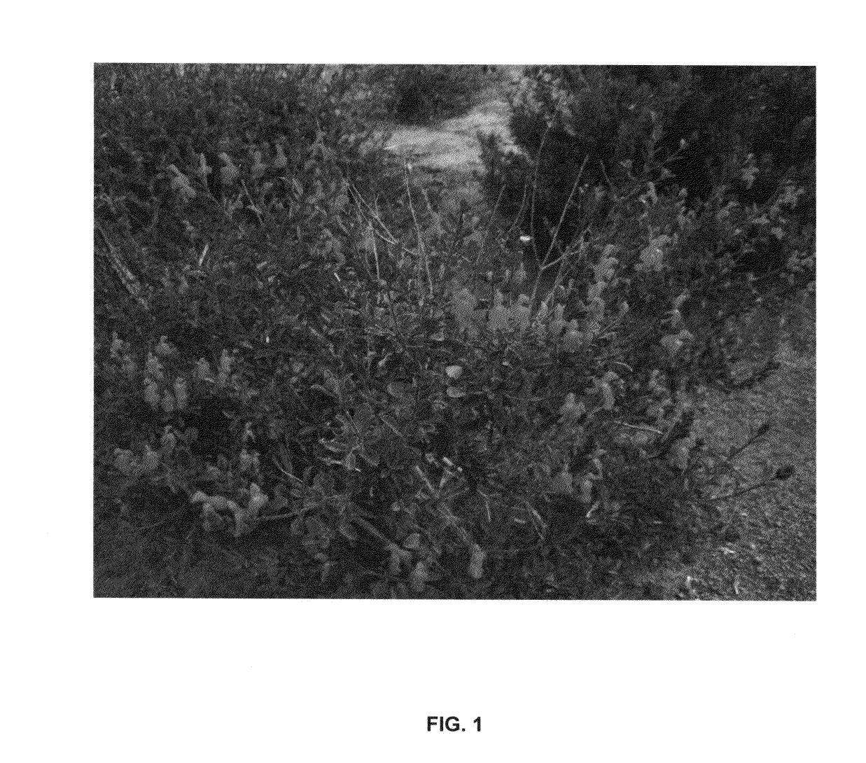

FIG. 2 shows a closeup of Salvia greggii ‘Ruby Slippers’ inflorescences and flowers.

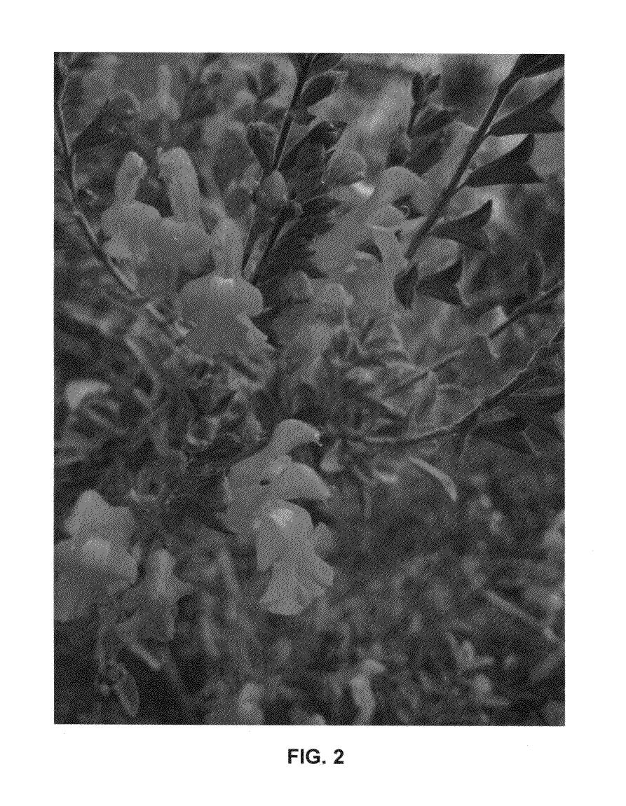

### DETAILED PLANT DESRIPTION

The following plant measurements were taken from a container grown specimen. The color descriptions are based upon the 5^{th} edition R.H.S. Colour Chart. Color names other than common usage are as listed in COLOR Universal Language and Dictionary of Names, by Kenneth L. Kelly and Deane B. Judd; National Bureau of Standards special publication 440. Washington, D.C. U.S. Department of Commerce, National Bureau of Standards, December 1976.

Salvia greggii ‘Ruby Slippers’ is a highly branched, suffrutescent shrub which grows to about 2.5 feet tall by 3.5 feet wide. The young stems are squarish, about 2-2.5 mm in cross section, the surface finely puberulent, the hairs caducous with age. Stem color 138B with barely visible striations terminally to being striped with 59A basally. Stems thicken with age to a more cylindrical shape and a relatively uniform 164D color. The older stems have papery, peeling bark. Stems range from about 2-15 mm in diameter, thickening with age. Internodes vary from 1-42 mm long, with paired, opposite leaves at the nodes. Branch angles vary from 35-45 degrees.

Leaves (color closest to 138A on both adaxial and abaxial surfaces) are opposite, and ovate/elliptical in shape, leaf base acute, apex rounded to obtuse. Leaf margins are entire basally, crenulate/denticulate apically. Leaf surface is adaxially lustrous, glabrous; abaxially less lustrous, glabrous. Leaf size is highly variable, from 4 mm wide×11 mm long up to 17 mm wide×27 mm long. Leaf veins are obscure and pinnate, slightly lighter than the surrounding leaf tissue, color 138B. Petioles range in length from 3-10 mm long, more or less half cylindrical in shape, the flat side held adaxially, color adaxially 138A, abaxially 138B.

Inflorescences are produced terminally, forming a square stemmed spikate raceme, axis color distally in area of flowering 59A, basally post flowering changing to 138B. The surface of the inflorescence axis is puberulent. Inflorescences grow from 12-26 cm long with approximately 15-20 nodes each. Floral bracts are carinate, 3-3.5 mm wide×5-7 mm long, ovate, apiculate, somewhat paleaceous and caducous several days before anthesis. Bract adaxial surface is glabrous, abaxial surface hispidulous. Bracts are striate, 7 nerved, the nerves and apex color 59A, basally 142D. Much of the internerval area is colored 141D, but the colors generally vary somewhat with growth conditions. Flowers are paired and opposite on the inflorescence. Flowers produced are typically 29 mm long×18 mm high×15-16 mm wide, tubular, bilabiate, ventricose with an overall color of 50A-B, the color lighter at the throat. Pedicels terete, 3 -4 mm long×0.5 mm in diameter. Pedicel color ranges from 60B near the floral attachment to 142B at the base. Pedicel surface is sparsely puberulent. The upper lip is 10 mm long×4 mm high×4 mm wide, hooded, the anterior portion covered with pilose hairs (the hairs colored 50A), basally glabrous, color 50A-B the stigma and stamens exserted from the tip. The interior (adaxial) upper lip is glabrous, 50D. The lower lip is spreading and somewhat reflexed, glabrous, more or less 4 lobed, entire and somewhat crisped, 12 mm high×15-16 mm wide, color 50A-50B with specular spotting, lighter near the throat 50D. The abaxial lower lip is glabrous 50A-B, grading to color 50D at the throat. The glabrous floral tube reaches 19mm long and is grooved. The pistil is about 24 mm long, the naked ovary 4 lobed, the lobes ovoid, 1 mm long×0.5 mm wide, color 1C or lighter attached to an expanded spheroidal receptacle, 1 mm diameter, color 1C or lighter. The stigma is unequally divided, the lobes parting dorsally, the upper 2.5 mm long, the lower 1 mm long, both narrowly acute, color 50A. A beard extends basally from about the midpoint of the upper lobe toward and below the fork about 4 mm. The combined stigma and style reach about 23 mm long the style elliptical, laterally compressed, 0.75 mm high×0.25 mm wide in cross section. The style color grades from that of the stigma basally to 50D or lighter. The 2 stamens are paired and epipetalous, the filaments fused at the attachment, then forming a united, detached, basal extension that forms a teeter totter like device that tips apically downward when a hummingbird inserts it bill into the tube, placing pollen on the bird's bill to pollinate the next flower. The forward portion of the filament measures 5 mm long×0.8 mm in diameter, tapering to the anther. The portion below the attachment measures 7 mm long×0.8 mm in diameter. The filaments are lighter than 50D. The dehisced anthers are basifixed, 2 mm long×1 mm thick, the pollen colored 26B.

The fused calyx is bilabiate, laterally compressed, at anthesis measuring 11 mm long×7 mm high×2.5 mm wide, striped and ridged, the ridges 59A, the valleys 143B, the exterior surface puberulent interior glabrous. Apex of upper lobe is apiculate, acute, the lower lobe acutely divided, 2 lobed, both lobes apiculate, the lobes unequally acute. Under local conditions, seed production apparently has not occurred with ‘Ruby Slippers’, so no seeds were examined.

### COMPARISONS TO RELATED SALVIA GREGGII

Compared to its female parent, ‘Lipstick’, ‘Ruby Slippers’ is considerably more heat tolerant, more compact, with the flower throat less white than that of ‘Lipstick’, which has a very white throat.

I claim:

1. A new and distinct Salvia greggii plant substantially as described and illustrated herein.
